(19) United States
(12) Patent Application Publication
(10) Pub. No.: US 2011/0217242 A1
(43) Pub. Date: Sep. 8, 2011

(54) Phenol with color

(76) Inventors: David Dercher, Radnor, PA (US); Suellen Dercher, Radnor, PA (US)

(21) Appl. No.: 12/932,601

(22) Filed: Mar. 2, 2011

Related U.S. Application Data
(60) Provisional application No. 61/339,169, filed on Mar. 2, 2010.

(51) Int. Cl. A61K 31/05; A61P 31/00; A61P 23/00

(52) U.S. Cl. 424/10.3

(57) ABSTRACT

1. Liquefied (or Liquified) Phenol USP (Carbolic Acid Liquefied) is Phenol maintained in a liquid condition by the presence of 10.0% water. Its topical medical use includes P & A (Phenol & Alcohol) procedures, and other procedures necessitating an anesthetic antiseptic, solvent, or cauterant. In treating ingrown nails, it is applied to the nail bed to prevent regrowth of nails. As a colorless solution, it can be difficult to see where an application is going during a procedure. Phenol with color eliminates this problem by creating a readily visible way of identifying where the application is going.

2. The patent calls for recognition of our innovation to mix Phenol with color for visual enhancement for physicians or others. “Phenol with color” is thus the innovation being patented and not the actual name of a product.

### CROSS-REF.

This application claims Priority 2: Provisional Patent Application No. 61/339,169; filing date Mar. 2, 2010.

### BACKGROUND & SUMMARY

1. Phenol is a clear liquid used topically for various medical procedures, including P & A (Phenol & Alcohol) procedures. It has been used as a topical anesthetic, antiseptic, and solvent. As a topical pharmaceutical manufacturer, our invention involves adding color to Phenol (or Phenol Solution) so that it is readily visible and identified by the user.

2. Phenol with color denotes our innovation and does not represent the name of the product we will be manufacturing.

### NOTE ON DRAWINGS

Not applicable. The patent is only for the use of color in Phenol Solution.

1. We claim an innovation of adding color to phenol to allow physicians or others to see exactly where the phenol application is going: This visual enhancement makes a procedure involving Phenol easier, safer, and more accurate.

2. This patent claim applies only to any color added to Phenol, and does not include packaging or applicator.
